(19) United States
(12) Patent Application Publication
(10) Pub. No.: US 2007/0156161 A1
(43) Pub. Date: Jul. 5, 2007

(54) Method and device for repositioning tissue

(76) Inventors: Kevin S. Weadock, Hillsborough, NJ (US); Parris Wellman, Hillsborough, NJ (US); James J. Rudnick, Mahwah, NJ (US); Jessica Liberatore, Marlboro, NJ (US)

Correspondence Address: BODNER & O'ROURKE, LLP, 425 BROADHOLLOW ROAD, SUITE 108, MELVILLE, NY, 11747, US

(21) Appl. No.: 11/322,144

(22) Filed: Dec. 29, 2005

(51) Int. Cl. A61B 17/32

(52) U.S. Cl. 606/170

(57) ABSTRACT

A device for repositioning tissue includes a hand held main body having a housing which defines a chamber for holding tissue. The chamber is connected to a negative pressure source to create a vacuum in the chamber. A flexible cannula is affixed to the main body, and includes a distal end and a proximate end, with openings through the thickness of the walls of each. The cannula defines a hollow lumen which is in communication with the distal end opening and the proximate end opening and the tissue holding chamber. The distal end of the cannula may be positioned in proximity to a patient's body to receive tissue therefrom through the distal end opening and to transport the tissue through the lumen and proximate end opening to the tissue holding chamber in response to the vacuum created in the tissue holding chamber.

### BACKGROUND OF THE INVENTION

1. Field of the Invention

The present invention relates generally to methods and devices for repositioning tissue, and more specifically relates to methods and devices used in liposuction. Even more particularly, the present invention relates to methods and devices used in facial lipoplasty and dermal contouring.

2. Description of the Prior Art

The normal aging process of humans leads to bony resorption, decreased tissue elasticity, and altered distribution of fat deposits. When combined with the sustained effects of gravity over time, these factors lead to ptosis of the cervicofacial support structures and the classic appearance of aging. Each individual's cervicofacial features are determined by the underlying bony and cartilaginous framework, and by the covering skin and soft tissue envelope. These characteristics are all ultimately determined at a genetic level but may be influenced to varying degrees by environmental factors (e.g., nutrition, exercise, aging, medications, toxin exposure, actinic damage, trauma, and surgery). As adverse environmental factors exert their toll, ptosis of facial support features causes drooping of malar and buccal fat pads. Loss of skin elasticity produces coarse and fine wrinkles and sagging of facial skin. Tissue laxity and the maldistribution of fat deposits lead to the formation of jowls and wattles, and redundant loose tissue hanging from the mandible and chin respectively. In the neck, accumulation of fat and ptosis of the platysma results in prominent banding producing the “turkey gobbler” appearance.

Materials such as collagen and hyaluronic acid are frequently used to augment dermal contour defects and fill wrinkles. Adipose tissue, i.e. fat, can also be used for this purpose. However, current liposuction cannulae, typically used for liposuction of areas such as the hips and abdomen, are too large to provide a safe, refined lipoplasty of the thigh to reposition within the face. Surgeons have therefore resorted to using regular syringes with pointed needles to suck fat out of the thigh to reposition it within the face. This is a cumbersome, unrefined and inexact method, incapable of providing the aesthetic quality that patients seek. Smaller cannulae have recently been developed for the face, but these are rigid and still too large to safely fit in the spaces of the face housing unwanted fat.

Many cosmetic surgeons transplant fat from other areas of the body into the face, where it acts as a filler of wrinkles and furrows. This transplantation process varies from one surgeon to another, and a loss of adipose tissue viability often occurs during the ex-vivo manipulations, such as syringe changes, vessel transfers and filtering.

There is currently in existence, to the knowledge of the inventors herein, no hand held system that can reproducibly and reliably perform subtle and refined lipoplasty of the face and neck without undue pain to the patient, and no means within such a system to maintain the viability of the transposed adipose tissue at acceptable levels.

### OBJECTS AND SUMMARY OF THE INVENTION

It is an object of the present invention to provide a method and device for safely and efficiently repositioning tissue that is particularly useful in lipoplasty of the human face and neck.

It is another object of the present invention to provide a hand held micro-lipoplasty device that can reproducibly and reliably perform subtle and refined lipoplasty of the face and neck without undue pain to the patient.

It is a further object of the present invention to provide a system for repositioning tissue which maintains the viability of the transposed adipose tissue at acceptable levels.

It is still another object of the present invention to provide a micro-lipoplasty system which achieves enhanced viability of adipocytes relative to conventional ex-vivo methods.

It is yet a further object of the present invention to provide a liposuction device which may be used to remove fat cells, fluid and other material from one site, such as a patient's leg, and transfer such material immediately to another site, such as the patient's face, without the need to transfer fat into another syringe or vessel.

It is still another object of the present invention to provide a lipoplasty device that is able to deliver therapeutic substances (e.g., anti-pain, anti-bacterial, lipolytics, electrolytes and vasoactives) into a patient while performing lipoplasty.

It is yet a further object of the present invention to provide a lipoplasty device which enables a surgeon to correct mistakes immediately.

It is also an object of the present invention to provide a method and device for repositioning tissue which allow for enhanced body contouring without cannula movement.

It is another object of the present invention to provide a micro-lipoplasty device which is small in scale for ergonomic efficiency.

It is yet another object of the present invention to provide a method and apparatus for repositioning tissue which could be used in the biopsy or transfer of tissues other than fat deposits, such as dermis, bone marrow, skeletal muscle, islet cells, uterus, lung tissue, and tumors.

In accordance with one form of the present invention, a device for repositioning tissue includes a hand holdable main body and a cannula extending from the main body. The main body has structure which defines a chamber for holding tissue therein. The chamber is in fluid communication with a source of negative pressure to create at least a partial vacuum therein.

The cannula has a distal end and a proximate end disposed axially opposite the distal end. The proximate end of the cannula is affixed to the main body. The cannula also has an opening formed in each of the distal end and the proximal end, and defines a hollow lumen extending at least partially along the axial length thereof. The lumen is in fluid communication with the tissue chamber and the cannula openings in the proximate and distal ends, which openings are further in fluid communication with each other.

The distal end of the cannula may be positioned in proximity to a patient's body to receive tissue therefrom through the distal end opening and to transport the tissue through the lumen and proximate end opening to the tissue chamber in response to the at least partial vacuum created in the tissue chamber.

In a preferred form of the invention, the structure which defines the tissue chamber includes a first portion and a second portion, at least a part of the second portion of which is telescopically receivable in the first portion to selectively provide the tissue chamber with a changeable or adjustable volume. Even more preferably, the second portion of the tissue chamber includes a free end receivable by the first portion and a plunger disposed on the free end, the free end and plunger of the second portion being reciprocatingly axially slidable within the first portion to selectively vary the volume of the tissue chamber.

Once tissue is withdrawn by forces comprised of at least the negative pressure source from a patient's body at one location, through the lumen and received by the tissue chamber of the main body, the physician or surgeon may relocate the distal end of the cannula at a different site to dispense tissue from the device by forcing the plunger of the second portion of the hand/holdable main body into the first portion through hand pressure to cause tissue held in the chamber to pass from the chamber through the cannula lumen and distal end opening to be dispensed at the different site.

In a further preferred form of the invention, the device may include a motor, a moveable shaft coupled to the motor which passes through the lumen of the cannula, and a cutting blade mounted on the distal end of the moveable shaft in alignment with the distal end opening in the cannula. The cutting blade will be exposed through the distal end opening so as to contact and excise tissue thereat. This cutting action, in concert with the negative pressure, facilitates rapid motility of tissue and fluids.

These and other objects, features and advantages of the present invention will be apparent from the following detailed description of illustrative embodiments thereof, which is to be read in connection with the accompanying drawings.

### BRIEF DESCRIPTION OF THE DRAWINGS

FIG. 1 is a cross-sectional view of an illustrative depiction of the tissue repositioning device formed in accordance with one form of the present invention.

FIG. 2 is a cross-sectional view of an illustrative depiction of the tissue repositioning device of the present invention shown in FIG. 1, which illustrates a different positional state of certain structure of the device from that which is shown in FIG. 1.

FIG. 2A is a cross-sectional view of an illustrative depiction of the tissue repositioning device of the present invention shown in FIGS. 1 and 2, with the addition of a cooling element.

FIG. 3 is a side view of the tissue repositioning device of one form of the present invention, with the housing wall thereof partially broken away to reveal internal components of the device.

FIG. 4 is a perspective view of the tissue repositioning device of the present invention in accordance with another form thereof.

FIG. 5 is a perspective view of a tissue repositioning device formed in accordance with an alternative form of the present invention.

FIG. 6 is one side view of the tissue repositioning device of the present invention shown in FIG. 5.

FIG. 7 is another side view of the tissue repositioning device of the present invention shown in FIG. 5.

### DETAILED DESCRIPTION OF THE PREFERRED EMBODIMENTS

The present invention is a device which is particularly suitable for micro-lipoplasty of a patient's face and neck and can store or transfer autologous adipose tissue without significant losses in the viability of the fraction of cells living within the tissue. The device of the present invention enables a surgeon to correct mistakes immediately or to perform very subtle, refined contouring of fat pads in the face, neck and around the eyes of a patient. The viability of adipose tissue may be maintained by the addition of various substances (e.g., nutrients, insulin, growth factors and the like) to a chamber within the system of the present invention that nourish adipocytes or assist the patient, such as providing to the chamber antimicrobials, anesthetics, vasoconstrictors and the like which, as will be described in greater detail, are injected using the system of the present invention into the patient at a particular site during lipoplasty.

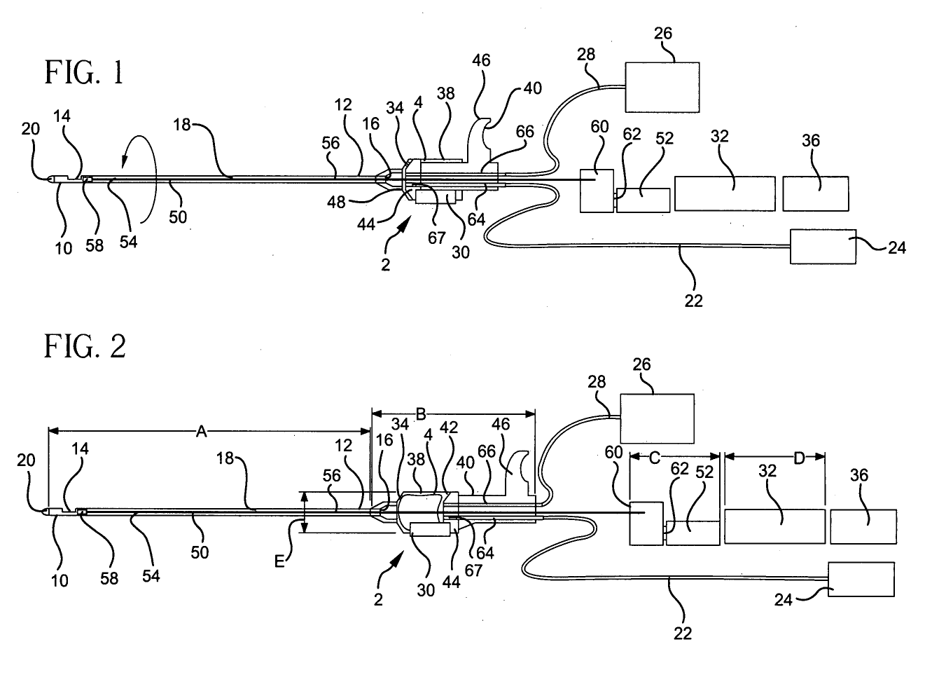

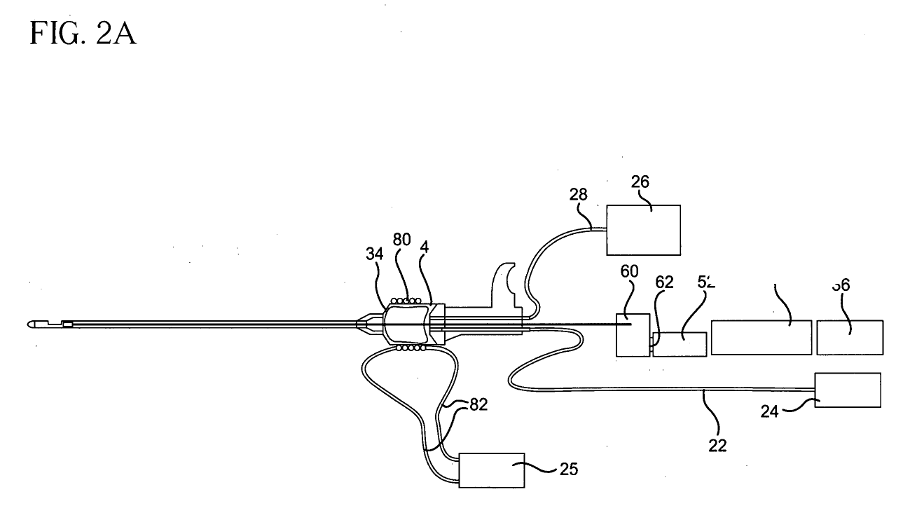

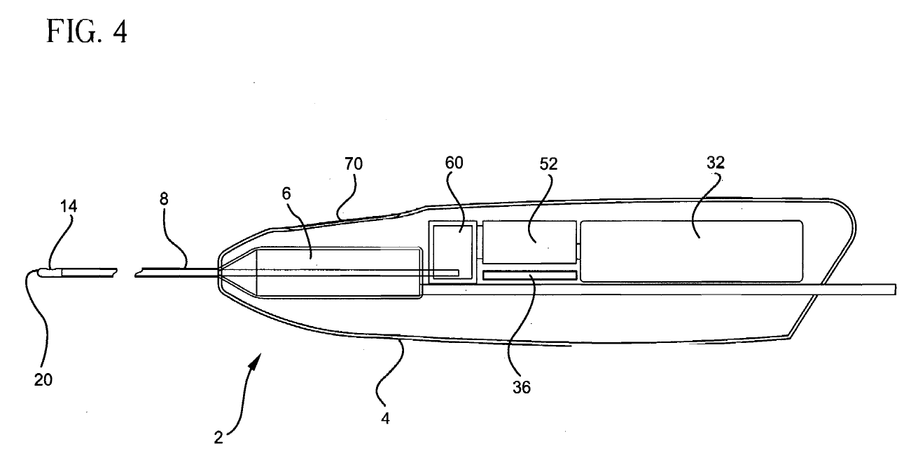

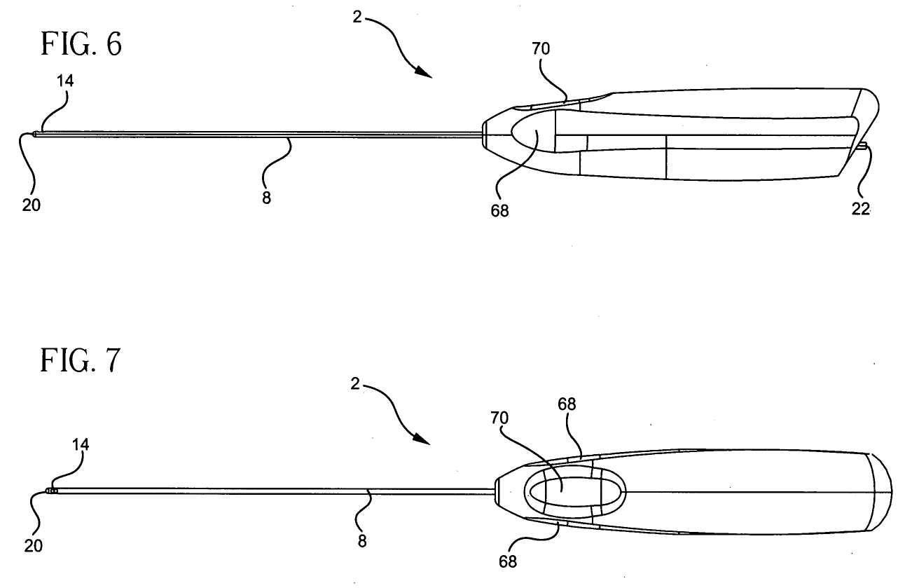

Referring now to FIGS. 1-7 of the drawings, it will be seen that a device for repositioning tissue formed in accordance with the present invention includes a hand held main body 2 having a housing 4 or other structure (e.g., a bellows or the like), that defines a chamber 6 in the interior thereof for holding tissue excised from a patient's body. The device also includes a cannula 8 extending from the main body 2 and connected thereto. The cannula 8 has a distal end 10 and a proximate end 12 which is disposed axially opposite the distal end 10. The proximate end 12 of the cannula is affixed to the main body 2. The cannula 8 has an opening 14, 16 formed in each of the distal end 10 and the proximate end 12, and further defines a hollow lumen 18 extending at least partially along the axial length thereof. The lumen 18 is in fluid communication with the tissue chamber 6 of the main body, and the cannula openings 14, 16 in the distal and proximate ends. Preferably, and as shown in FIGS. 1 and 2, the distal end opening 14 is in the form of a notch cut into the sidewall of the cannula 8.

Preferably, the cannula 8 is semi-rigid or at least partially flexible so that the physician may position the distal end thereof at a particular site on the patient's body where tissue is to be removed or repositioned. In a preferred embodiment, the cannula 8 is made from Nitinol and has a rounded, atraumatic tip 20. The Nitinol cannula 8 facilitates a flexible, gentle movement by the surgeon or physician and reduces the risk of injuring vital facial structures such as nerves and blood vessels. The outer diameter of the cannula 8 preferably should be less than 1.5 mm (millimeters). The lumen diameter of the cannula 8 preferably should be large enough to transport adipose tissue without having a deleterious mechanical effect on adipocyte viability. A preferred range for the lumen diameter is about 0.25 mm to about 0.50 mm. Other acceptable materials from which the cannula 8 may be made include 316 L stainless steel or a dense plastic, such as polycarbonate, Teflon, or polymethyl methacrylate (PMMA).

Preferably, the tissue holding chamber 6 is capable of storing about one to about ten cubic centimeters volume of fat or other fluid. As mentioned previously, the tissue holding chamber 6 is in fluid communication with the cannula lumen 18 and receives tissue and other fluids that are transported through the distal end opening 14 of the cannula, the lumen 18 and the proximate end opening 16 to the tissue chamber 6. In order to remove tissue from the patient's body and to transport the tissue through the cannula lumen 18 to the tissue chamber 6 of the hand held main body 2, preferably the tissue chamber is in fluid communication via a tube or conduit 22 with a source of negative pressure 24 in order to create at least a partial vacuum in the chamber 6. Also, the chamber 6 is preferably in fluid communication with a source of therapeutic substance 26. Therapeutic substances include epinephrine, anesthetics such as Lidocaine™, saline, antibiotics, lipolytic substances, growth factors, cytokines, antioxidants, free radial scavengers, and others. Other fillers such as collagen, hyaluronic acid, polylactic acid, polyglycolic acid, polyglycolide acid and other biomaterials may also be added to the tissue for tissue filling, or cell attachment purposes. The biomaterials can be milled to particle sizes of 50-200 microns before being mixed with the tissue. The substances are contained within a source 26 outside the hand held unit but in fluid communication with the chamber 6 via a tube or conduit 28. The substances can be used to nourish the fat cells within the chamber or they can be used to treat the patient for pain, etc., as they can be administered by the surgeon or physician from the tissue chamber 6 of the hand held main body 2, through the lumen 18 and out the distal end opening 14 of the cannula to be dispensed at a particular site on the patient's body.

In another preferred embodiment of the present invention, and as shown in FIGS. 1 and 2 of the drawings, the device of the present invention for repositioning tissue may further include a heating element 30. The heating element 30 is in thermal communication with the tissue chamber 6 to provide heat to the tissue chamber. Even more specifically, the heating element 30 maintains the tissue chamber 6 at a predetermined temperature. The heating element 30 may be a resistive element or flexible film heater situated near or at least partially about the chamber defining structure. The resistive element 30 is coupled to a power source, such as a battery 32, also preferably situated within the hand held main body 2 of the device, and by a temperature sensor 34 also situated in thermal communication with the tissue chamber 6. The temperature sensor 34 and resistive element 30 are preferably connected to a microprocessor 36, also preferably situated within the hand held main body 2 of the device. The temperature sensor 34 senses the temperature within the tissue chamber 6 and provides a signal indicative of the temperature in the tissue chamber to the microprocessor 36. The microprocessor 36 selectively controls power provided to the resistive element 30 to maintain the temperature within the tissue chamber 6 at a predetermined temperature. Preferably, the chamber 6 is held at 37 degrees C. As a result, the viability of transplanted tissue is improved.

Means for cooling the tissue may be used if cooling of the tissue is desired. For example, and as shown in FIG. 2A, a coolant may be circulated through a tubular coil 80 helically wrapped about a portion of the housing 4 in proximity to the tissue chamber 6 to cool the tissue held therein in order to maintain its viability. The coil 80 is connected to a source of pressurized coolant 25 by flexible conduits 82. The temperature sensor 34, described previously, will maintain tissue chamber 6 at a desired temperature, and will directly or indirectly control the source of pressurized coolant 25 to circulate or not circulate, or provide or not provide, coolant to the coil 80.

In another form of the present invention, and again as shown in FIGS. 1 and 2 of the drawings, it will be seen that the device for repositioning tissue includes a tissue chamber 6 that has a changeable or adjustable volume. More specifically, the structure defining the tissue chamber, which could be the housing 4 of the main body 2, is preferably collapsible and expandable to receive a varying amount of tissue in the chamber 6 and to dispense tissue from the chamber held thereby so that the tissue may be transplanted from one site on the patient's body, such as the hips, to another site, such as the face. A collapsible and expandable bellows type structure defining the tissue chamber 6 therein is envisioned to be within the scope of the present invention. Another structure defining the tissue chamber 6 with an adjustable volume is where the housing 4 of the main body 2 is formed of at least a first portion 38 and a second portion 40, at least a part of which is telescopically receivable in the first portion 38. Such structure selectively provides the tissue chamber 6 with an adjustable volume.

Even more specifically, the second portion 40 of the tissue chamber defining structure, such as the housing 4, includes a free end 42 receivable by the first portion 38, and a plunger 44 disposed on the free end 42. The plunger 44 preferably makes a fluidtight seal with the walls of first portion 38 to contain tissue, fluids and other material within the tissue chamber 6 and to allow the free end 42 of the second portion 40 of the housing 4 to be reciprocatingly slidable axially within the first portion 38 in order to selectively vary the volume of the tissue chamber. The transverse or cross-sectional dimensions of the mating first portion 38 and the second portion 40 of the main body housing 4 may be rectangular or circular.

The second portion 40 of the housing 4 may include a projection 46 radially extending outwardly therefrom, which serves as a finger grip in order to dispense tissue and fluid contained in the tissue chamber 6 when the surgeon or physician is transplanting tissue to another site on the patient's body or when he is administering a therapeutic substance to the patient. The physician or surgeon would place his thumb against the finger grip 46 and his second and third fingers against the front surface 48 of the main body 2 with the cannula 8 between them to force tissue or therapeutic substances contained in the chamber 6 from the chamber through the proximal end opening 16 of the cannula, through the lumen 18 and out the distal end opening 14 to a selected site on the patient's body.

The tissue repositioning system and device of the present invention also preferably includes a moveable shaft 50 which is disposed at least partially within the lumen 18 of the cannula 8. The shaft 50 is in mechanical communication with a source of mechanical movement, such as a motor 52 which is preferably contained within the hand held main body 2, and moved within the lumen 18 in response to the motor 52. The shaft 50 has a distal end 54 which is situated in proximity to the distal end 10 of the cannula 8, and a proximate end 56 which is axially opposite the distal end 54 and situated in proximity to the proximate end 12 of the cannula.

A cutting blade 58 is mounted on and affixed to the distal end 54 of the moveable shaft 50. The cutting blade 58 is positioned in alignment with the distal end opening 14 in the cannula, and is moveable within the lumen 18 at the distal end opening in response to movement of the shaft 50. The cutting blade 58 is exposed through the distal end opening 14 to contact and excise tissue from the patient. The cutting action provided by the blade 58, in concert with the negative pressure, facilitates rapid motility of adipose tissue.

The micro-lipoplasty system and device of the present invention may further include gearing 60 (e.g., a gear box), the gearing 60 being coupled to the motor shaft 62 and the moveable shaft 50 within the lumen 18. Preferably, the gearing 60 is also situated on the hand held main body 2 of the device. The gearing 60 may be reduction gearing, to reduce the rotational velocity of the motor shaft 62 to a lower velocity at which the moveable shaft 50 in the lumen and cutting blade 58 attached thereto rotate, or alternatively, may increase the effective rotational velocity of the motor shaft 62 to a greater speed at which the cutting blade 58 and lumen shaft 50 rotate. As a further alternative, the moveable shaft 50 in the lumen and cutting blade 58 may reciprocatingly slide axially within the lumen 18, and the gearing 60 may translate the rotational movement of the motor shaft 62 to the reciprocatingly sliding movement of the cutting blade 58 and lumen shaft 50.

Preferably, the moveable shaft 50 in the lumen 18 extends through the tissue chamber 6 and the plunger 44 and is coupled to the gearing 60. A bore through the plunger 44 is dimensioned to closely receive but allow rotational or reciprocating movement of the lumen shaft 50 to effect a cutting motion of the blade 58 at the distal end opening 14 of the cannula. The plunger 44 also preferably has two further extension tubes 64, 66 which are closely received in bores formed through the thickness thereof, which tubes 64, 66 are respectively connected to a flexible conduit 22 connected to a source of negative pressure 24 and another flexible conduit 28 connected to a source of therapeutic substance 26. The inside bores of the tubes 64, 66 which pass through the plunger 44 communicate with the tissue chamber 6 to provide therapeutic substances and a negative pressure to the tissue chamber. Also, preferably a screen 67 is positioned at the plunger end of extension tube 64 which is connected to the negative pressure source 24. The screen 67 acts to filter any non-adipose material from that which collects in the tissue chamber 6 by the negative pressure created through the screen.

The system and device of the present invention is capable of moving fat from one site of the body to another, e.g., from the hips to the face. In this system, as opposed to conventional ex-vivo manipulations currently practiced, all of the functions of thermal regulation, filtering off of non-adipose material, administering therapeutics, etc. can all be performed within and from the chamber 6. This allows for increased viability of the adipose tissue being transplanted. When the device of the present invention is used in fat transplantation, the physician or surgeon is provided with a refined and subtle means by the present invention of delivering high aesthetic value to the patient's face. The device of the present invention also enables a physician or surgeon to correct any mistakes immediately, since the device and system can add, remove or transplant tissue at very small volumes, for example, less than 1 cubic centimeter. The device and system of the present invention also permit enhanced contouring of the patient's body without cannula movement, thus sparing the patient any undue pain and injury. Also, with reference to FIG. 2 of the drawings, one form of the device has dimensions A, B, C, D and E which are 6, 3.06, 1.69, 1.88 and 0.75 inches, respectively.

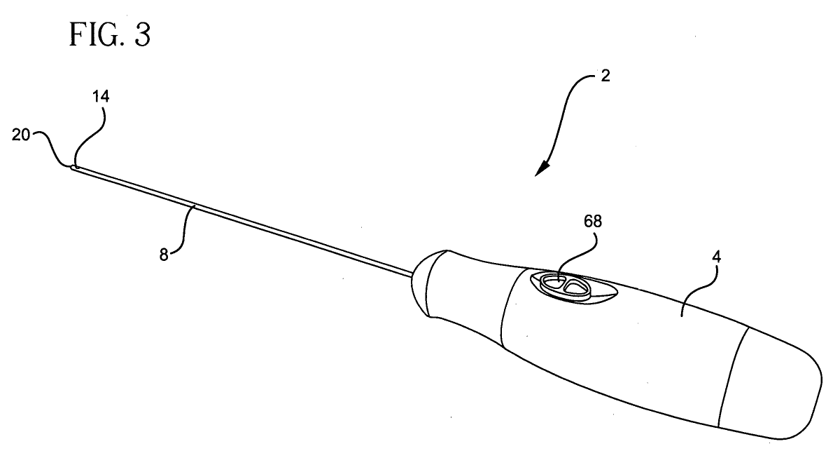

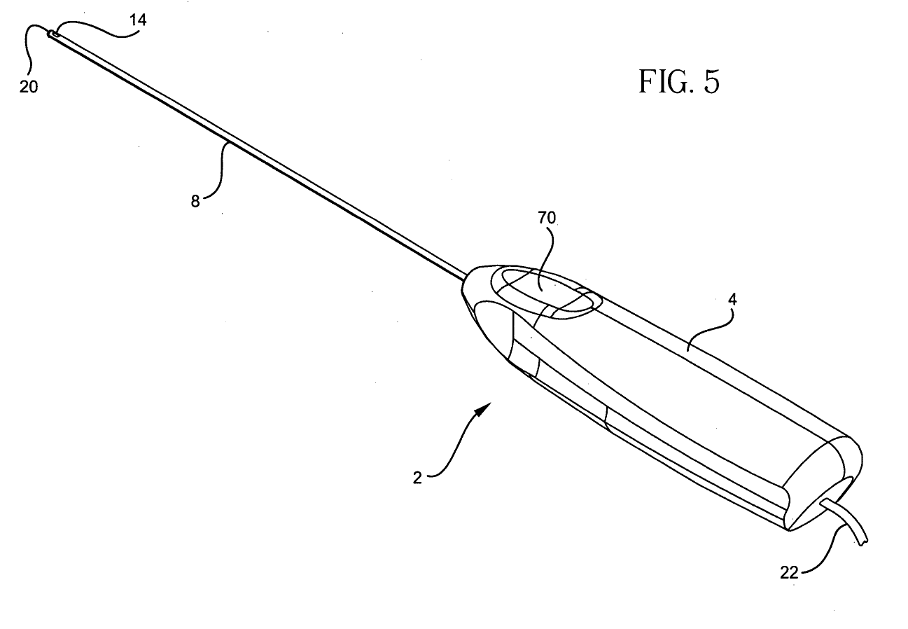

FIGS. 3-7 show various embodiments of the device for repositioning tissue formed in accordance with the present invention and depict the ergonomic features of the device as well as a preferred positioning of various components within the hand held main body 2. As shown in the drawings, the cannula 8 is attached to the hand held main body 2 of the device at its proximal end 12. The main body of the device is designed to ergonomically fit within the physician's or surgeons' hand so that it is comfortable and easy to manipulate and position strategically at a desired site on the patient's body. FIGS. 5-7 illustrate the device with one or more actuation pushbutton switches 68 which control power to one or more of the heating element 30, the motor 52 and the source of negative pressure 24, if control lines or wires are provided from the hand held main body to the negative pressure source. Preferably, the actuation pushbutton switches 68 may be positioned on opposite sides of the housing 4 of the hand held main body 2.

Also, a window 70 formed of transparent material may be mounted on the housing 4 in alignment with the tissue chamber 6 and within an opening formed in the housing so that the quality and volume of the material being processed and contained in the tissue chamber may be visibly assessed by the physician or surgeon.

As can be seen from the foregoing description, the device for repositioning tissue of the present invention enables tissue repositioning to be performed on a patient and is particularly suitable for surgical procedures of the face and neck. The device can store or transfer autologous adipose tissue without significant losses in the viability of the cells living within the tissue. The device enables a surgeon or physician to correct mistakes immediately or enables the surgeon to perform very subtle, refined contouring of fat pads in the patient's face, neck and around the eyes. The viability of adipose tissue may be maintained by the addition of various therapeutic substances to the tissue chamber 6 within the hand held main body 2 that nourish adipocytes or assists the patient. While performing the lipoplasty, the physician or surgeon will be able to deliver therapeutic substances, for example, anti-pain, anti-bacterial and other substances, back into the patient while performing lipoplasty. The viability of transplanted adipose tissue will also be improved by the ability of the device to maintain the collected tissue in the chamber 6 at a predetermined temperature.

The device of the present invention may be used to remove fat cells from one site, such as the patient's leg, and place the fat cells immediately back into another site, such as the patient's face. The device of the present invention also allows for enhanced contouring of the patient's body without cannula movement.

The hand held main body 2 of the lipoplasty device of the present invention is small in scale for ergonomic efficiency. The device may also be used to biopsy or transfer tissues other than fat cells, such as dermis, tumors or the like.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

What is claimed is:

1. A device for repositioning tissue, which comprises:
a handholdable main body, the main body having structure defining a chamber for holding tissue therein, the chamber being fluid communicatable with a source of negative pressure to create at least a partial vacuum therein; and
a cannula having a distal end and a proximate end disposed axially opposite the distal end, the proximate end of the cannula being affixed to the main body, the cannula having an opening formed in each of the proximate end and the distal end thereof and defining a hollow lumen extending at least partially along the axial length thereof, the lumen being in fluid communication with the tissue chamber and the cannula openings in the distal and proximate ends;
whereby the distal end of the cannula may be positioned in proximity to a patient's body to receive tissue therefrom through the distal end opening and to transport the tissue through the lumen and proximate end opening to the tissue chamber in response to the at least partial vacuum created in the tissue chamber.

2. A device for repositioning tissue as defined by claim 1, wherein the main body further includes a heating element, the heating element being in thermal communication with the tissue chamber to provide heat to the tissue chamber.

3. A device for repositioning tissue as defined by claim 1, wherein the main body further includes a cooling element, the cooling element being in thermal communication with the tissue chamber to provide a cooling temperature to the tissue chamber.

4. A device for repositioning tissue as defined by claim 1, wherein the main body further includes a temperature control element, the temperature control element being in thermal communication with the tissue chamber to maintain the tissue chamber at a predetermined temperature.

5. A device for repositioning tissue as defined by claim 4, wherein the main body further includes a temperature sensor, the temperature sensor being in thermal communication with the tissue chamber to sense the temperature thereof, the temperature control element being responsive to the temperature sensor and adjusting the temperature of the tissue chamber in response thereto.

6. A device for repositioning tissue as defined by claim 2, wherein the volume of the tissue chamber is changeable.

7. A device for repositioning tissue as defined by claim 6, wherein the structure defining the tissue chamber is collapsible and expandable to receive a varying amount of tissue in the chamber and to dispense from the chamber tissue held thereby.

8. A device for repositioning tissue as defined by claim 7, wherein the tissue chamber defining structure includes a housing of the main body, the housing being formed of at least a first portion and a second portion at least a part of which is telescopically receivable in the first portion to selectively provide the tissue chamber with a changeable volume.

9. A device for repositioning tissue as defined by claim 8, wherein the second portion of the tissue chamber defining structure includes a free end receivable by the first portion and a plunger disposed on the free end, the free end and plunger of the second portion being reciprocatingly axially slidable within the first portion to selectively vary the volume of the tissue chamber.

10. A device for repositioning tissue as defined by claim 9, whereby movement of the plunger of the second portion in one direction axially within the first portion forces tissue held in the tissue chamber from the chamber and through the cannula lumen and distal end opening to be dispensed thereby.

11. A device for repositioning tissue as defined by claim 1, wherein the tissue chamber is in fluid communication with a source of therapeutic substance.

12. A device for repositioning tissue as defined by claim 1, wherein the cannula is at least partially flexible.

13. A device for repositioning tissue as defined by claim 1, which further comprises a moveable shaft disposed at least partially within the lumen of the cannula, the shaft being in mechanical communication with a source of mechanical movement and moving within the lumen in response to the source of mechanical movement, the shaft having a distal end situated in proximity to the distal end of the cannula, and a proximate end axially opposite the distal end and situated in proximity to the proximate end of the cannula, and further comprising a cutting blade, the cutting blade being disposed at the distal end of the moveable shaft and in alignment with the distal end opening, the cutting blade being movable within the lumen at the distal end opening in response to movement of the shaft and being exposable through the distal end opening to contact and excise tissue thereat.

14. A device for repositioning tissue as defined by claim 13, wherein the energy source of mechanical movement is a motor, the motor being mounted on the hand/holdable main body.

15. A device for repositioning tissue as defined by claim 14, which further comprises gearing, the gearing being coupled to the motor and the moveable shaft.

16. A device for repositioning tissue as defined by claim 15, wherein the motor generates rotational movement at a first rotational velocity, and wherein the gearing causes the shaft to rotate in at least a second rotational velocity in response to the rotational movement generated by the motor, the second rotational velocity of the shaft being different from the first rotational velocity generated by the motor.

17. A device for repositioning tissue as defined by claim 15, wherein the motor generates rotational movement, and wherein the gearing causes the shaft to reciprocatingly slide axially within the lumen in response to the rotational movement generated by the motor.

18. A method for repositioning tissue, which comprises the steps of:
generating at least a partial vacuum in a tissue holding chamber of a handholdable device;
excising tissue from a patient's body at one location with a moving cutting blade affixed to a shaft moveable within a cannula, the cannula having a distal end and a proximate end which is disposed axially opposite the distal end, and defining a hollow lumen extending at least partially along the axial length thereof, the lumen being in fluid communication with the tissue chamber and the cannula openings in the distal and proximate ends; and
transporting the tissue excised by the cutting blade through the lumen and proximate end opening to the tissue chamber in response to the at least partial vacuum created in the tissue chamber.

19. A method of repositioning tissue as defined by claim 18, which further comprises the steps of:
repositioning the distal end of the cannula at a second location on the patient's body, the second location being different from the first location; and
forcing tissue held in the tissue chamber from the chamber and through the cannula lumen and distal end opening so that the tissue is dispensed and repositioned at the second location on the patient's body.
